(19) United States
(12) Patent Application Publication
(10) Pub. No.: US 2005/0075403 A1
(43) Pub. Date: Apr. 7, 2005

(54) Ambroxol for the treatment of inflammation in the pharynx

(75) Inventors: Anke Esperester, Mainz (DE); Jean-Michel Vix, Wiesbaden (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim Pharma GmbH & Co. KG, Ingelheim (DE)

(21) Appl. No.: 10/677,967

(22) Filed: Oct. 2, 2003

(51) Int. Cl. A61K 31/137

(52) U.S. Cl. 514/641

(57) ABSTRACT

The invention relates to the use of ambroxol (trans-4-(2-amino-3,5-dibromobenzylamino)-cyclohexanole) and the pharmacologically acceptable salts thereof for preparing a pharmaceutical composition for the treatment of inflammation in the pharynx.

### BACKGROUND TO THE INVENTION

The invention relates to the use of ambroxol ( trans-4-(2-amino-3,5-dibromobenzylamino)-cyclohexanole) and the pharmacologically acceptable salts thereof for preparing a pharmaceutical composition for the treatment of inflammation in the pharynx.

Anti-inflammatory agents for relieving pain in the pharynx often have the drawback of side effects, e.g. in the form of gastrointestinal disturbances, allergies and local irritations in the case of topical preparations. No anti-inflammatory effect in the pharynx is known using pharmaceutical compositions containing exclusively conventional local anaesthetics as active ingredients like lidocaine and benzocaine.

It has been pre-clinically and clinically documented that ambroxol has a clear local anaesthetic and pain relieving effect.

The in vitro effect of ambroxol on the release and synthesis of cytokines involved in inflammatory diseases of the bronchopulmonary tract is described in the prior art.

There are many cases where substances which have shown a particular anti-inflammatory effect in vitro but did not show the effect in vivo.

Ambroxol was shown to decrease the secretion of interleukin-2 (IL-2) and interferon-γ (INF-γ) by bronchoalveolar lavage cells and peripheral blood mononuclear cells stimulated with phythemagglutine (Pfeifer S, Zissel G, Kienast K, Muller-Quernheim J. Eur J Med Res 1997;2:129-132). IL-2 and INF-γ play a role in the course of chronic inflammation in the bronchoalveolar region. In a further study, ambroxol was found to inhibit the production of the cytokines IL-1 and tumor necrosis factor α (TNF-α) in human mononuclear cells stimulated with lipopolysaccharide (Bianchi M, Mantovani A, Erroi A, Dinarello C A, Ghezzi P. Agents Actions 1990;31:275-27). IL-1 and TNF-α are inflammatory mediators associated with pulmonary damage and lung fibrosis.

The effects seen in the aforementioned studies were interpreted as an anti-inflammatory effect of ambroxol.

However, these results are contradictory to the in vitro findings of other authors who stated that ambroxol appears to enhance inflammatory responses through shifting the local balance of anti-inflammatory IL-10 and inflammatory IL-12 to IL-12 dominance (Aihara M, Dobashi K, Akiyama M, Naruse I, Nakazawa T, Mori M. Respiration 2000;67:662-671).

There are other examples that demonstrate that an in vitro effect on cytokine regulation does not correlate with the effects seen in vivo. For instance NSAIDs such as ketoprofen, were found to induce the release of inflammatory TNF in vitro, but otherwise demonstrated clinical efficacy as anti-inflammatory compounds (Ghezzi P, Melillo G, Meazza C, Sacco S, Pellegrini L, Asti C, Porzio S, Marullo A, Sabbatini V, Caselli G, Bertini R. J Pharmacol Exp Ther 1998;287:969-974). No definite correlation could also be made between in vivo anti-inflammatory animal data and in vitro inhibition of lipoxygenase/cyclogenase of compounds such as isoflavanes (Montandon J B, Zijlstra F J, Wilson J H, Grandjean E M, Cicurel L. Int J Tissue React 1989;11:107-112).

The aim of the present invention is to prepare a well-tolerated active substance for the treatment of inflammation in the pharynx.

### SUMMARY OF THE INVENTION

The invention relates to pharmaceutical compositions comprising ambroxol (trans-4-(2-amino-3,5-dibromobenzylamino)-cyclohexanole) and the pharmacologically acceptable salts thereof and methods using such compositions for the treatment of inflammation and the reduction of redness in the pharynx.

### BRIEF DESCRIPTION OF THE FIGURE

FIG. 1. Redness of the Pharyngeal Mucosa, Post Treatment. The results of three experiments examining the inflammatory effect of ambroxol lozenges by measurement of the redness symptom and 2) the efficacy and tolerability of ambroxol lozenges relative to placebo in relieving the symptoms of sore throat of at least moderately severe intensity in patients suffering from oro-pharyngeal catarrh accompanied by pain are shown.

### DESCRIPTION OF THE INVENTION

Surprisingly, it has been found that, when administered locally, ambroxol has an anti-inflammatory effect on the pharyngeal mucosa.

The invention therefore relates to the use of ambroxol or one of the pharmacologically acceptable salts thereof for preparing a pharmaceutical composition for the local treatment of inflammation in the pharynx.

The invention further relates to the use of a pharmaceutical composition containing ambroxol for preparing a medicament for the local treatment of inflammation in the pharynx.

Preferably the invention relates to the use of a pharmaceutical composition, wherein the single dose contains 15 to 50 mg of ambroxol, preferably in form of its hydrochloride salt, most preferably 20 mg of ambroxol hydrochloride.

More preferably the invention relates to the use of a solid, suckable or slowly dissolving formulation of a pharmaceutical composition, preferably to the use of lozenges.

Particularly preferred is the use of a liquid formulation of a pharmaceutical composition in the form of a spray or gargle.

Further particularly preferred is the use of a pharmaceutical composition consisting of ambroxol hydrochloride, a flavouring, a lubricant, a matrix material, a sweetening agent and a polyethyleneglycol.

The invention further relates to the use of a suckable tablet containing ambroxol based on sugar alcohols as the matrix material, wherein it contains a pharmaceutically acceptable layered silicate and a polyethyleneglycol, optionally together with other pharmaceutical excipients, taste or flavouring agents for preparing a medicament for treating inflammation in the pharynx.

The invention further relates to the use of ambroxol for preparing a pharmaceutical composition for the reduction of redness in the throat associated with pharyngitis.

The invention further relates to the use of a pharmaceutical composition for preparing a medicament for the reduction of redness in the throat associated with pharyngitis.

Acids suitable for forming salts of ambroxol include for example hydrochloric acid, hydrobromic acid, sulphuric acid, phosphoric acid, nitric acid, oxalic acid, malonic acid, fumaric acid, maleic acid, tartaric acid, citric acid, ascorbic acid and methanesulphonic acid, preferably hydrochloric acid.

Ambroxol may be used on its own or combined with other pharmacologically active substances. It may be applied in any of the preparation forms which are suitable for local use. Preparations suitable for sucking or dissolving slowly in the mouth include, for example, tablets or sweets based on sugar or sugar substitutes or pastille-like products with a gum arabic or gelatine base.

Suitable solutions for spraying, gargling and rinsing include aqueous preparations, advantageously with the addition of viscosity-increasing substances such as modified celluloses, acrylic acid derivatives or polyvinyl compounds.

In addition, the liquid forms in particular may contain sweetening agents and moisture retainers such as glycols and sugar alcohols, for example.

All the forms are flavoured in the conventional way, e.g. by the addition of ethereal oils.

The preparations may be produced by methods known in pharmacy.

### EXAMPLES

The examples are intended solely to illustrate the invention and are not to be regarded as limiting.

The activity of ambroxol according to the invention is intended to be illustrated by the following three examples of clinical trials which investigate: 1) the inflammatory effect of ambroxol lozenges by measurement of the redness symptom and 2) the efficacy and tolerability of ambroxol lozenges relative to placebo in relieving the symptoms of sore throat of at least moderately severe intensity in patients suffering from oro-pharyngeal catarrh accompanied by pain.

### Example 1

The first study was a multi-centre, prospective, placebo-controlled, randomized, double-blind trial involving two days of treatment with up to six lozenges containing 20 mg ambroxol hydrochloride per day.

Besides the primary endpoint, pain, which was reduced statistically significantly, also the assessment of the redness of the pharyngeal mucosa was assessed at baseline and at day two. 109 patients were treated with ambroxol and 109 patients with placebo.

At baseline there was no difference between the active treatment group and placebo; at visit two (after two days of treatment), in contrast, there was less redness in the active treatment group compared to placebo (p-value: 0.026) for ambroxol lozenges vs. placebo.

Two other confirmatory clinical trials were performed to investigate the efficacy and tolerability of ambroxol lozenges at doses of 20 mg ambroxol hydrochloride relative to placebo in the same indication as in the first trial. The design was similar for both trials: multi-centre, prospective, placebo-controlled, randomized, double-blind trials involving three days of treatment with up to six lozenges containing 20 mg ambroxol hydrochloride per day.

### Example 2

In one study 111 patients were treated with ambroxol lozenges 20 mg whilst 108 patients were treated with placebo. At visit one there was no difference between the active treatment and placebo; at visit two (i.e. after three days of treatment) in contrast, there was less redness in the active treatment group compared to placebo (p-value: 0.010).

### Example 3

In the other study 128 patients were treated with ambroxol lozenges (20 mg) while 127 patients were treated with placebo. The results regarding redness were similar to that of the former trial. At visit two there was less redness in the active treatment group compared to placebo (p-value: 0.009).

As a result of the three confirmatory clinical trials the efficacy of ambroxol lozenges (20 mg) has been documented regarding pain relief in sore throat and decrease of redness of the pharyngeal mucosa. Pain and redness are two major symptoms of inflammation. Although the relieve of pain could at least partly be regarded as the local anaesthetic effect of ambroxol, by the decrease of redness ambroxol lozenges were clearly proven to feature anti-inflammatory properties clinically, in sore throat. This had not been demonstrated for the substance ambroxol before.

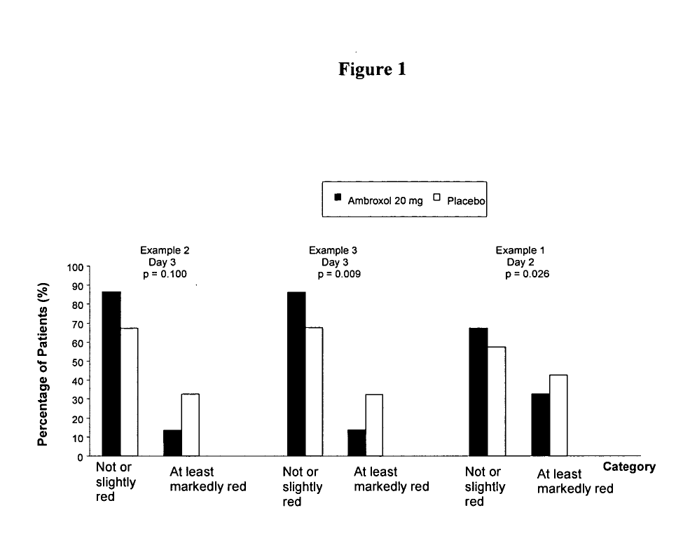

FIG. 1 shows the percentage of patients in relation to the redness of the throat for all three studies.

The following examples of pharmaceutical formulations illustrate the present invention without restricting its scope:

Formulation 1

| Suckable pastille | per pastille |
|---|---|
| ambroxol hydrochloride | 20.0 mg |
| peppermint flavouring | 16.0 mg |
| sorbitol | 1373.5 mg |
| saccharin sodium | 0.5 mg |
| Macrogol 6000 | 30.0 mg |
| talc | 60.0 mg |

Formulation 2

| Suckable pastille | per tablet |
|---|---|
| Ambroxol hydrochloride | 20.0 mg |
| Lysozyme hydrochloride | 5.0 mg |
| Dipotassium glycyrrhizinate | 2.5 mg |
| Cetylpyridinium Chloride | 1.0 mg |
| Chlorpheniramine Maleate | 1.0 mg |
| Xylitol | 920.5 mg |
| D-Mannitol | 9.5 mg |
| Polyvinylpyrrolidone | 21.0 mg |
| Stearic acid | 10.0 mg |
| Peppermint oil | 6.0 mg |
| light anhydrous silicic acid | 1.0 mg |
| talc | 1.0 mg |
| magnesium stearate | 1.5 mg |

Formulation 3

| Spray or gargle | g/100 g |
|---|---|
| Ambroxol hydrochloride | 1.0 g |
| Sorbitol | 30.0 g |
| Glycerol | 10.0 g |
| Ethanol | 5.0 g |
| I-Menthol | 0.01 g |
| Peppermint oil | 0.06 g |
| Saccharine | 0.03 g |
| Water | 53.9 g |

All publications and patents cited herein are incorporated by reference in their entireties.

1. A pharmaceutical composition comprising ambroxol or one of the pharmacologically acceptable salts thereof for the local treatment of inflammation in the pharynx.

2. The pharmaceutical composition according to claim 1, wherein a single dose contains 15 to 50 mg of ambroxol.

3. The pharmaceutical composition according to claim 1, which is in the form of a solid, suckable or slowly dissolving formulation.

4. The pharmaceutical composition according to claim 1, which is a liquid formulation in the form of a spray or gargle.

5. The pharmaceutical composition according to claim 1 for the reduction of redness in the throat associated with pharyngitis.

6. A pharmaceutical composition comprising ambroxol based on sugar alcohols as the matrix material; a pharmaceutically acceptable layered silicate; and a polyethyleneglycol, optionally together with other pharmaceutical excipients, taste or flavouring agents.

7. A method for treating inflammation in the pharynx comprising administering a pharmaceutical composition comprising ambroxol or one of the pharmacologically acceptable salts thereof for the local treatment of inflammation in the pharynx.

8. The method according to claim 7 wherein the pharmaceutical composition consists of ambroxol hydrochloride, a flavouring, a lubricant, a matrix material, a sweetening agent and a polyethyleneglycol.

9. The method according to claim 7 wherein the pharmaceutical composition is in the form of a suckable tablet, and wherein the tablet comprises ambroxol based on sugar alcohols as the matrix material; a pharmaceutically acceptable layered silicate; and a polyethyleneglycol, optionally together with other pharmaceutical excipients, taste or flavouring agents.

10. A method for the reduction of redness in the throat comprising administering a pharmaceutical composition comprising ambroxol or one of the pharmacologically acceptable salts thereof such that redness in the throat is reduced.

11. A method for the reduction of redness in the throat comprising administering a pharmaceutical composition comprising ambroxol based on sugar alcohols as the matrix material; a pharmaceutically acceptable layered silicate; and a polyethyleneglycol, optionally together with other pharmaceutical excipients, taste or flavouring agents such that redness in the throat is reduced.
